(19) United States
(12) Patent Application Publication
(10) Pub. No.: US 2012/0277483 A1
(43) Pub. Date: Nov. 1, 2012

(54) Water Control In Alcohol Production From Hydrogenation

(75) Inventors: Trinity Horton, Houston, TX (US); Victor J. Johnston, Houston, TX (US); Adam Orosco, Houston, TX (US); Lincoln Sarager, Houston, TX (US); Mark O. Scates, Houston, TX (US); Manuel Salado, Seabrook, TX (US); Nathan Kirk Powell, Waxahachie, TX (US); Fred Ronald Olsson, Corpus Christi, TX (US)

(73) Assignee: Celanese International Corporation, Dallas, TX (US)

(21) Appl. No.: 13/292,870

(22) Filed: Nov. 9, 2011

Related U.S. Application Data
(63) Continuation-in-part of application No. 13/094,641, filed on Apr. 26, 2011.

(51) Int. Cl. C07C 29/147; C07C 29/80

(52) U.S. Cl. 568/884

(57) ABSTRACT

Recovery of alcohol, in particular ethanol, from a crude product obtained from the hydrogenation of a feed stream comprising a carbonylation stock selected from the group consisting of acetic acid, and acetic acid and an ester thereof. The hydrogenation reaction produces a reactor product that is dehydrated to produce a dried product stream that comprises less water than the feed stream. This controls the additional water fed to the hydrogenation unit.

### CROSS REFERENCE TO RELATED APPLICATIONS

This application claims priority to U.S. application Ser. No. 13/094,641, filed on Apr. 26, 2011, the entire contents and disclosures of which are incorporated herein by reference.

### FIELD OF THE INVENTION

The present invention relates generally to processes for producing alcohol and, in particular, to controlling the water during alcohol production from hydrogenation of acids, aldehydes, and/or esters.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulose materials, such as corn or sugar cane. Conventional methods for producing ethanol from petrochemical feed stocks, as well as from cellulose materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulose material, are converted to ethanol by fermentation. However, fermentation is typically used for consumer production of ethanol, which is suitable for fuels or human consumption. In addition, fermentation of starchy or cellulose materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. During the reduction of alkanoic acid, e.g., acetic acid, other compounds are formed with ethanol or are formed in side reactions. These impurities limit the production and recovery of ethanol from such reaction mixtures. For example, during hydrogenation, esters are produced that together with ethanol and/or water form azeotropes, which are difficult to separate. In addition when conversion is incomplete, unreacted acetic acid remains in the reactor product, which must be removed to recover ethanol. Also, the hydrogenation of acetic acid typically yields ethanol and water along with small amounts of side reaction-generated impurities and/or by-products. At maximum theoretical conversion and selectivity, the reactor product would comprise approximately 72 wt. % ethanol and 28 wt. % water. In order to form purified ethanol, much of the water that is co-produced must be removed from the reactor product. In addition, when conversion is incomplete, unreacted acid may remain in the reactor product. It is typically desirable to remove this residual acetic acid from the reactor product to yield purified ethanol.

EP02060553 describes a process for hydrogenating ethanoic acid to ethanol and for reducing the proportion of ethyl ethanoate by co-feeding water.

Ethanol recovery systems for other types of ethanol production processes are also known. For example, U.S. Pub. No. 2008/0207959 describes a process for separating water from ethanol using a gas separation membrane unit. The gas separation membrane unit may be used to remove water from a fermentation broth that has been partially dewatered, for example by one or more of a distillation column or molecular sieves. Additional systems employing membranes and distillation columns are described in U.S. Pat. Nos. 7,732,173; 7,594,981; and 4,774,365, the entireties of which are incorporated herein by reference. See also Huang, et al, “Low-Energy Distillation-Membrane Separation Process,” Ind. Eng. Chem. Res., Vol. 40 (2010), pg. 3760-68, the entirety of which is incorporated herein by reference.

The need remains for improved processes for recovering ethanol from a reactor obtained by reducing alkanoic acids, such as acetic acid, and/or mixtures with other carbonyl group-containing compounds, such as ethyl acetate.

### SUMMARY OF THE INVENTION

In a first embodiment, the present invention is directed to a process for producing ethanol from a feed stream comprising water and a carbonylation stock selected from the group consisting of acetic acid, and acetic acid and an ester thereof. In one embodiment, the feed stream is water and acetic acid. The process further comprises hydrogenating the acetic acid and/or an ester thereof from the feed stream in a reactor to form a reactor product comprising ethanol, water, and one or more organic impurities, separating at least a portion of the reactor product to yield a water stream and a dried product, wherein the dried product comprises less water based on total weight than the feed stream, and separating at least a portion of the dried product to yield one or more streams comprising the one or more organic impurities and an ethanol stream comprising less than 0.01 wt. % of the one or more organic impurities.

In a second embodiment, the present invention is directed to a process for producing ethanol, comprising providing a feed stream comprising water and a carbonylation stock selected from the group consisting of acetic acid, and acetic acid and an ester thereof; hydrogenating the acetic acid and/or an ester thereof from the feed stream in a reactor to form a reactor product comprising ethanol, water, and one or more organic impurities; separating at least a portion of the reactor product to yield a water stream and a dried product, wherein the dried product comprises less water based on total weight than the feed stream; and recovering ethanol from the dried product. In one embodiment, the ethanol remains in the liquid phase when recovering ethanol from the dried product.

### BRIEF DESCRIPTION OF DRAWINGS

The invention may be more completely understood in consideration of the following detailed description of various embodiments of the invention in connection with the accompanying drawings, wherein like numerals designate similar parts.

FIG. 1 is a schematic diagram of an ethanol production system having a water separation unit comprising membranes in accordance with one embodiment of the present invention.

FIG. 2 is a schematic diagram of an ethanol production system having a water separation unit comprising a PSA unit in accordance with one embodiment of the present invention.

FIG. 3 is a schematic diagram of an ethanol production system having a combined distillation and membrane separation system with two distillation columns in accordance with one embodiment of the present invention.

FIG. 4 is a chart of conversion and selectivity results from an exemplary process of the present invention.

### DETAILED DESCRIPTION OF THE INVENTION

### Introduction

The present invention relates to processes for producing alcohol by hydrogenating feed comprising an acid and/or an ester thereof, and water in the presence of a catalyst. For purposes of the present invention, the process is described for producing ethanol and the feed stream may comprise water and a carbonylation stock selected from the group consisting of acetic acid, and acetic acid and an ester thereof. In one embodiment, the feed stream is water and acetic. The hydrogenation reaction produces a reactor product that comprises ethanol, water, and one or more organic impurities. The organic impurities may be selected from the group consisting of ethyl acetate, acetic acid, acetone, diethyl acetal, diethyl ether, and acetaldehyde.

Including water in the feed stream would be expected to be detrimental to ethanol production because water is a co-product of the reaction and is not converted during hydrogenation. In the hydrogenation of acetic acid, water is co-produced with ethanol in about a 1:1 molar ratio. However, surprisingly and unexpectedly, it has been found that feeding acetic acid and/or a mixture of acetic acid and ethyl acetate, in combination with water to a hydrogenation reactor does not substantially influence the conversion of acetic acid and/or ethyl acetate to ethanol. This allows the present invention to use less pure starting feed streams as well as recycle streams that may contain water. The presence of water in the feed stream advantageously may allow the use of different grades of acetic acid other than glacial acetic acid as a feedstock. Acetic acid may be taken from a carbonylation facility that does not require drying the acetic acid to remove the water, or which employs a smaller or lower energy drying column that allows some water to remain in the acetic acid. Ethyl acetate may be taken from an esterification facility that does not require drying the ethyl acetate to remove the water, or which employs a smaller or lower energy drying technique that allows some water to remain in the ethyl acetate. In addition, no pre-dehydration of the feed stream is necessary, thus reducing energy and capital costs.

Typically, feed streams are dehydrated and then the ethanol product is dehydrated to remove the co-produced water. The present invention may advantageously reduce the dehydrating steps and dehydrate the reactor product instead of the feed streams. This reduces the processing steps and may simplify the ethanol production process.

In some embodiments, water may be combined with glacial acetic acid to form the feed stream. Optionally, glacial acetic acid may be mixed with dilute acetic acid, i.e., vinegar, which may be produced from fermentation. The water may be obtained from an outside source or from a stream separated from the reactor product. It is preferred that a portion of the water may be obtained from a stream separated from the reactor product.

In one embodiment, the feed stream comprises water in amounts of up to 25 wt. %, e.g., up to 20 wt. % water, or up to 10 wt. % water. Thus, the total cumulative water in the reactor may be up to 25 wt. %, based on the total contents of the reactor. In terms of ranges the feed stream may comprise from 0.001 wt. % to 25 wt. % water, e.g., from 2 wt. % to 20 wt. %, from 0.5 to 15 wt. %, or from 4 wt. % to 10 wt. %. The remaining portion of feed stream preferably comprises acetic acid and/or ethyl acetate, and hydrogen. The molar ratio of hydrogen to acetic acid and/or ethyl acetate may be from about 100:1 to 1:100, e.g., from 50:1 to 1:50, from 20:1 to 1:2, or from 12:1 to 1:1. The molar ratio of hydrogen to acetic acid preferably is greater than 2:1, e.g., greater than 4:1 or greater than 8:1. The molar ratio of hydrogen to ethyl acetate preferably is greater than 2:1, e.g., greater than 5:1 or greater than 10:1.

Due to the presence of water in the feed, the reactor product contains more water than would otherwise be produced by hydrogenation acids and/or esters. In one embodiment, the process advantageously separates the reactor product to yield a water stream and a dried product. In one embodiment, the process removes at least 90% of the water from the reactor product, e.g., at least 95% of the water or at least 97% of the water. In terms of ranges, the process may remove from 90% to 99.9% of the water from the reactor product, e.g., from 95% to 99% or from 97% to 99%. Preferably, the water is removed before any appreciable amount of organic impurities, including acetic acid and/or ethyl acetate, are removed from the reactor product. Removing water as an initial separation step beneficially results in an energy savings because the water is not carried through the process as the organic impurities are removed. In addition, initially dehydrating the reactor product may yield an ethanol product that has an acceptable water concentration for industrial and/or fuel applications. Thus, the number of process steps is reduced to control water concentrations when recovering ethanol.

The dried product comprises less water based on total weight than the feed stream. Depending on the water concentration in the feed stream, the dried product may have a water concentration that varies from 0.0005 wt. % to 20 wt. %, e.g., from 0.001 to 10 wt. %, or from 0.01 to 4 wt. %. In one embodiment, the dried product may comprise less than the azeotropic amount of water for an ethanol-water composition. The present invention produces a dried product that comprises less water than the feed stream to allow for efficient separation and recovery of the ethanol. In one embodiment, the dried product may be separated to yield one or more streams comprising the one or more organic impurities and an ethanol stream comprising less than 0.01 wt. % of the one or more organic impurities. The ethanol stream may have a suitable impurity level for an industrial and/or fuel application. In another embodiment, the reduced water concentration of the dried product may allow ethanol to be recovered by remaining in the liquid phase and not boiling the ethanol overhead in a distillation column. Keeping the ethanol in the liquid phase during recovery allows for reduced energy and/or capital requirements.

In the water removal step, the water may be separated from a reactor product with an adsorption unit, one or more membranes, molecular sieves, or a combination thereof. Preferably, the majority of the water in the initial water separation step is not removed using a distillation column. Suitable adsorption units include pressure swing adsorption (PSA) units and thermal swing adsorption (TSA) units. The adsorption units may comprise molecular sieves, such as aluminosilicate compounds. The use of adsorption units and/or membranes provides a low energy alternative to distillation columns for separating water from ethanol. In addition, adsorption units and/or membranes may be able to break the ethanol-water azeotrope to remove more water than a typical distillation column.

If an adsorption unit is employed for water removal, the adsorption unit may employ a suitable adsorption agent such as zeolite 3A or 4A. In one preferred embodiment, the adsorption unit comprises a pressure swing adsorption (PSA) unit that is operated at a temperature from 30° C. to 160° C., e.g., from 80° C. to 140° C., and a pressure of from 0.01 kPa to 550 kPa, e.g., from 1 kPa to 150 kPa. The PSA unit may comprise from two to five beds. In one embodiment, the reactor product is fed to a PSA to produce a water stream and a dried product.

In some embodiments, the reactor product is fed to a membrane or an array of membranes. The water preferably permeates across the membranes producing a dried product as a retentate stream. In this context, it should be appreciated that the term “dry” is used relative to the stream entering the membrane system as the retentate stream may comprise water. Suitable membranes include acid resistant membranes that have increased water permeability, i.e., high selectivities for permeating water. In one embodiment, the membrane may be a pervaporation membrane. The membrane may comprise a polymeric membrane, for example, comprising polyimide hollow fibers. Alternatively, the membrane may be a zeolite membrane or a hybrid membrane with both organic and inorganic components. The membrane or membranes preferably comprise one or more pervaporation membranes. Suitable membranes include shell and tube membrane modules having one or more porous material elements therein. Non-porous material elements may also be included. The material elements may comprise a polymeric element such as polyvinyl alcohol, cellulose esters, and perfluoropolymers. Membranes that may be employed in embodiments of the present invention include those described in Baker, et al., “Membrane separation systems: recent developments and future directions,” (1991) pages 151-169, and Perry et al., “Perry's Chemical Engineer's Handbook,” 7th ed. (1997), pages 22-37 to 22-69, the entire contents and disclosures of which are hereby incorporated by reference.

It should be noted that one or more membranes may be used in series or in parallel in order to achieve the desirable purity of the final ethanol product. In addition, it should be noted that either the permeate and/or the retentate stream may pass through additional membranes. Also a stream may be recycled through the same membrane to remove undesirable materials. For example, if it is desirable to obtain a dried product with a reduced amount of water, the reactor product stream may be fed through a first water permeable membrane. Then, the retentate stream may be fed through a second water permeable membrane to yield a second retentate stream. A portion of the second permeate stream may be recycled and combined with the reactor product to capture additional ethanol.

Since the water stream that is removed from the reactor product may be purged from the reaction system, it is preferred that the water stream comprises substantially no ethanol, e.g., less than 5000 wppm ethanol, or less than 500 wppm ethanol or less than 50 wppm ethanol. In order to ensure that a minimal amount of ethanol, if any at all, is removed from the system with the water stream, it may be desirable to use highly selective membranes, such as zeolite membranes. Highly selective membranes that minimize the amount of organics, including ethanol, that pass through the membrane in the permeate stream are preferred. In addition to water, the water stream may comprise acetic acid, and/or other organics, e.g., ethyl acetate or acetaldehyde, which would not be expected to significantly impact the amount of ethanol that is removed in the water stream. In some embodiments, when an array of membranes is used or a portion of the water stream is fed to another column, the membrane may be less selective for water and the water stream may contain up to 30 wt. % ethanol.

The dried product may be further processed to recover ethanol. Depending on conversion and selectivity in the reactor, the dried product also may comprise acetic acid and organics in addition to ethanol. One or more distillation columns may be used to remove these components. In addition, the remaining water may also be removed from the dried product and/or a derived ethanol stream, using an adsorption unit, one or more membranes, molecular sieves, extractive column distillations, or a combination thereof.

### Hydrogenation Process

The process of the present invention may be used with any hydrogenation process for producing ethanol. The materials, catalysts, reaction conditions, and separation processes that may be used in the hydrogenation of acetic acid are described further below.

The raw materials, acetic acid and hydrogen, used in connection with the process of this invention may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259 and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the reactor product may be separated from syngas. The syngas, in turn, may be derived from a variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into synthesis gas, and U.S. Pat. No. 6,685,754, which discloses a method for the production of a hydrogen-containing gas composition, such as a synthesis gas including hydrogen and carbon monoxide, are incorporated herein by reference in their entireties.

The feed to the hydrogenation reaction may comprise acetic acid, or mixtures of acetic acid and ethyl acetate. In some embodiments, the feed may also comprise other carboxylic acids and anhydrides, as well as acetaldehyde and acetone. Preferably, a suitable feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. As indicated, water may also be present in the acetic acid feed or separately fed to the hydrogenation reactor.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the ethanol synthesis reaction zones of the present invention without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

Some embodiments of the process of hydrogenating acetic acid to form ethanol may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation reaction may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa, or from 100 kPa to 1500 kPa. The reactants may be fed to the reactor at a gas hourly space velocity (GHSV) of greater than 500 hr⁻¹, e.g., greater than 1000 hr⁻¹, greater than 2500 hr⁻¹ oreven greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Contact or residence time can also vary widely, depending upon such variables as amount of acetic acid, catalyst, reactor, temperature, and pressure. Typical contact times range from a fraction of a second to more than several hours when a catalyst system other than a fixed bed is used, with preferred contact times, at least for vapor phase reactions, of from 0.1 to 100 seconds, e.g., from 0.3 to 80 seconds or from 0.4 to 30 seconds.

The hydrogenation of acetic acid to form ethanol is preferably conducted in the presence of a hydrogenation catalyst. Suitable hydrogenation catalysts include catalysts comprising a first metal and optionally one or more of a second metal, a third metal or any number of additional metals, optionally on a catalyst support. The first and optional second and third metals may be selected from Group IB, IIB, IIIB, IVB, VB, VIB, VIIB, VIII transition metals, a lanthanide metal, an actinide metal or a metal selected from any of Groups IIIA, IVA, VA, and VIA. Preferred metal combinations for some exemplary catalyst compositions include platinum/tin, platinum/ruthenium, platinum/rhenium, palladium/ruthenium, palladium/rhenium, cobalt/palladium, cobalt/platinum, cobalt/chromium, cobalt/ruthenium, cobalt/tin, silver/palladium, copper/palladium, copper/zinc, nickel/palladium, gold/palladium, ruthenium/rhenium, and ruthenium/iron. Exemplary catalysts are further described in U.S. Pat. No. 7,608,744 and U.S. Pub. No. 2010/0029995, the entireties of which are incorporated herein by reference. In another embodiment, the catalyst comprises a Co/Mo/S catalyst of the type described in U.S. Pub. No. 2009/0069609, the entirety of which is incorporated herein by reference.

In one embodiment, the catalyst comprises a first metal selected from the group consisting of copper, iron, cobalt, nickel, ruthenium, rhodium, palladium, osmium, iridium, platinum, titanium, zinc, chromium, rhenium, molybdenum, and tungsten. Preferably, the first metal is selected from the group consisting of platinum, palladium, cobalt, nickel, and ruthenium. More preferably, the first metal is selected from platinum and palladium. In embodiments of the invention where the first metal comprises platinum, it is preferred that the catalyst comprises platinum in an amount less than 5 wt. %, e.g., less than 3 wt. % or less than 1 wt. %, due to the high commercial demand for platinum.

As indicated above, in some embodiments, the catalyst further comprises a second metal, which typically would function as a promoter. If present, the second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel. More preferably, the second metal is selected from tin and rhenium.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 to 20 wt. %, e.g., from 0.1 to 10 wt. %, or from 0.1 to 5 wt. %. For catalysts comprising two or more metals, the two or more metals may be alloyed with one another or may comprise a non-alloyed metal solution or mixture.

The preferred metal ratios may vary depending on the metals used in the catalyst. In some exemplary embodiments, the mole ratio of the first metal to the second metal is from 10:1 to 1:10, e.g., from 4:1 to 1:4, from 2:1 to 1:2, from 1.5:1 to 1:1.5 or from 1.1:1 to 1:1.1.

The catalyst may also comprise a third metal selected from any of the metals listed above in connection with the first or second metal, so long as the third metal is different from the first and second metals. In preferred aspects, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal preferably is from 0.05 to 4 wt. %, e.g., from 0.1 to 3 wt. %, or from 0.1 to 2 wt. %.

In addition to one or more metals, in some embodiments of the present invention the catalysts further comprise a support or a modified support. As used herein, the term “modified support” refers to a support that includes a support material and a support modifier, which adjusts the acidity of the support material.

The total weight of the support or modified support, based on the total weight of the catalyst, preferably is from 75 wt. % to 99.9 wt. %, e.g., from 78 wt. % to 97 wt. %, or from 80 wt. % to 95 wt. %. In preferred embodiments that utilize a modified support, the support modifier is present in an amount from 0.1 wt. % to 50 wt. %, e.g., from 0.2 wt. % to 25 wt. %, from 0.5 wt. % to 15 wt. %, or from 1 wt. % to 8 wt. %, based on the total weight of the catalyst. The metals of the catalysts may be dispersed throughout the support, layered throughout the support, coated on the outer surface of the support (i.e., egg shell), or decorated on the surface of the support.

As will be appreciated by those of ordinary skill in the art, support materials are selected such that the catalyst system is suitably active, selective and robust under the process conditions employed for the formation of ethanol.

Suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof

As indicated, the catalyst support may be modified with a support modifier. In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, Co₂O₃, and Bi₂O₃.

In another embodiment, the support modifier may be a basic modifier that has a low volatility or no volatility. Such basic modifiers, for example, may be selected from the group consisting of: (i) alkaline earth oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

A preferred silica support material is SS61138 High Surface Area (HSA) Silica Catalyst Carrier from Saint Gobain NorPro. The Saint-Gobain NorPro SS61138 silica exhibits the following properties: contains approximately 95 wt. % high surface area silica; a surface area of about 250 m²/g; a median pore diameter of about 12 nm; average pore volume of about 1.0 cm³/g as measured by mercury intrusion porosimetry and a packing density of about 0.352 g/cm³ (22 lb/ft³).

A preferred silica/alumina support material is KA-160 silica spheres from Sud Chemie having a nominal diameter of about 5 mm, a density of about 0.562 g/ml, an absorptivity of about 0.583 g H₂O/g support, a surface area of about 160 to 175 m²/g, and a pore volume of about 0.68 ml/g.

The catalyst compositions suitable for use with the present invention preferably are formed through metal impregnation of the modified support, although other processes such as chemical vapor deposition may also be employed. Such impregnation techniques are described in U.S. Pat. Nos. 7,608,744 and 7,863,489 and U.S. Pub. No. 2010/0197485 referred to above, the entireties of which are incorporated herein by reference.

In particular, the hydrogenation of acetic acid may achieve favorable conversion of acetic acid and favorable selectivity and productivity to ethanol. For purposes of the present invention, the term “conversion” refers to the amount of acetic acid in the feed that is converted to a compound other than acetic acid. Conversion is expressed as a mole percentage based on acetic acid in the feed. The conversion may be at least 10%, e.g., at least 20%, at least 40%, at least 50%, at least 60%, at least 70% or at least 80%. Although catalysts that have high conversions are desirable, such as at least 80% or at least 90%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. Preferably, the catalyst selectivity to ethoxylates is at least 60%, e.g., at least 70%, or at least 80%. As used herein, the term “ethoxylates” refers specifically to the compounds ethanol, acetaldehyde, and ethyl acetate. Preferably, the selectivity to ethanol is at least 80%, e.g., at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 100 grams of ethanol per kilogram of catalyst per hour, e.g., at least 400 grams of ethanol per kilogram of catalyst per hour or at least 600 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram of catalyst per hour or from 600 to 2,000 grams of ethanol per kilogram of catalyst per hour.

Operating under the conditions of the present invention may result in ethanol production on the order of at least 0.1 tons of ethanol per hour, e.g., at least 1 ton of ethanol per hour, at least 5 tons of ethanol per hour, or at least 10 tons of ethanol per hour. Larger scale industrial production of ethanol, depending on the scale, generally should be at least 15 tons of ethanol per hour, preferably at least 30 tons of ethanol per hour. In terms of ranges, for large scale industrial production of ethanol, the process of the present invention may produce 15 to 160 tons of ethanol per hour, e.g., 30 to 80 tons of ethanol per hour. Ethanol production from fermentation, due the economies of scale, typically does not permit the single facility ethanol production that may be achievable by employing embodiments of the present invention.

In various embodiments of the present invention, the reactor product produced by the hydrogenation process, before any subsequent processing, such as purification and separation, will typically comprise ethanol, water, and one or more organic impurities. Exemplary compositional ranges for the reactor product are provided in Table 1. The “others” identified in Table 1 may include, for example, esters, ethers, aldehydes, ketones, alkanes, and carbon dioxide.

TABLE 1
REACTOR PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 70 | 15 to 70 | 15 to 50 | 25 to 50 |
| Acetic Acid | 0 to 90 | 1 to 80 | 15 to 75 | 20 to 70 |
| Water | 5 to 60 | 15 to 60 | 20 to 60 | 20 to 40 |
| Ethyl Acetate | 0 to 35 | 0 to 15 | 1 to 12 | 3 to 10 |
| Acetaldehyde | 0 to 10 | 0 to 3 | 0.1 to 3 | 0.2 to 2 |
| Others | 0.1 to 10 | 0.1 to 6 | 0.1 to 4 | Not |
| | | | | detectable |

In one embodiment, the reactor product comprises acetic acid in an amount less than 20 wt. %, e.g., less than 15 wt. %, less than 10 wt. % or less than 5 wt. %. In terms of ranges, the acetic acid concentration of Table 1 may range from 0.1 wt. % to 20 wt. %, e.g., 0.2 wt. % to 15 wt. %, from 0.5 wt. % to 10 wt. % or from 1 wt. % to 5 wt. %. In embodiments having lower amounts of acetic acid, the conversion of acetic acid is preferably greater than 75%, e.g., greater than 85% or greater than 90%. In addition, the selectivity to ethanol may also be preferably high, and is preferably greater than 75%, e.g., greater than 85% or greater than 90%.

### Ethanol Production

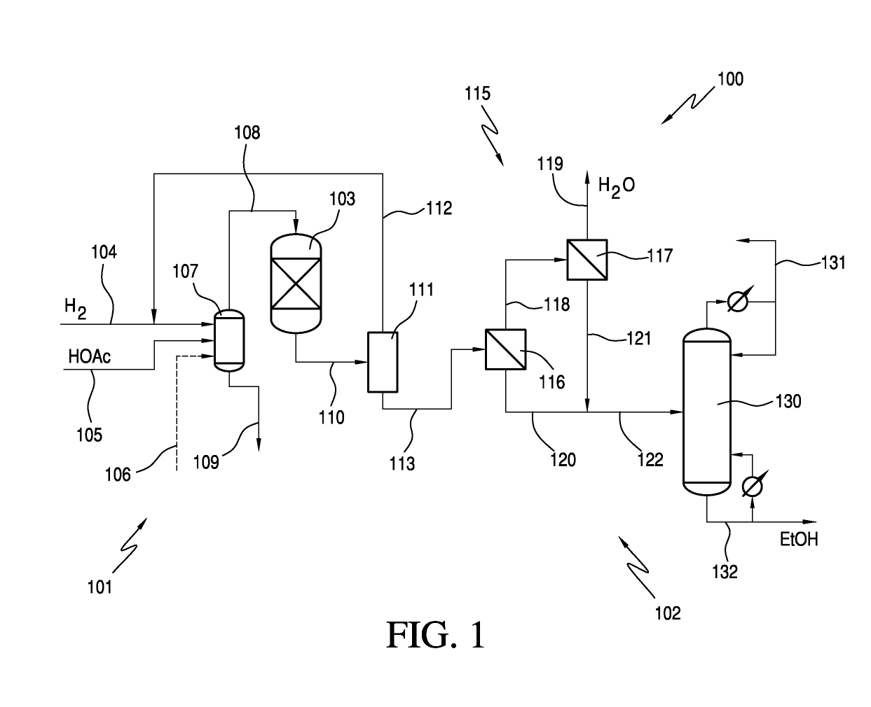

Exemplary ethanol recovery systems in accordance with embodiments of the present invention are shown in FIGS. 1-3. Each hydrogenation system 100 provides a suitable hydrogenation reactor and a process for separating ethanol from the reactor product according to an embodiment of the invention. For purposes of illustration, FIGS. 1-3 use acetic acid as the carbonylation stock in the feed to the hydrogenation reactor 103. Other embodiments the carbonylation stock may comprise mixtures of acetic acid and ethyl acetate.

System 100 comprises reaction zone 101 and separation zone 102. Reaction zone 101 comprises reactor 103, hydrogen feed line 104, acetic acid feed line 105, and optional water feed line 106. In some embodiments, acetic acid feed line 105 may comprise water in an amount of up to 25 wt. %. In other embodiments, optional water feed line 106 may be combined with acetic acid feed line 105 to increase the total amount of water fed to vaporizer 110 up to 25 wt. %. In addition, water feed line 106 may comprise an acetic acid recycle stream comprising acetic acid and water, derived from separation zone 102.

Hydrogen, acetic acid, and water are fed to a vaporizer 107 via lines 104 and 105, and optional line 106, respectively, to create a vapor feed stream in line 108 that is directed to reactor 103. In one embodiment, lines 104 and 105, and optional line 106, may be combined and jointly fed to vaporizer 107. The temperature of the vapor feed stream in line 108 is preferably from 100° C. to 350° C., e.g., from 120° C. to 310° C. or from 150° C. to 300° C. Any feed that is not vaporized is removed from vaporizer 107 and may be discarded via blowdown stream 109. In addition, although line 108 is shown as being directed to the top of reactor 103, line 108 may be directed to the side, upper portion, or bottom of reactor 103. Further modifications and additional components to reaction zone 101 and separation zone 102 are described below.

Reactor 103 contains the catalyst that is used in the hydrogenation of the carboxylic acid, preferably acetic acid, and esters thereof. In one embodiment, one or more guard beds (not shown) may be used upstream of the reactor to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens. During the hydrogenation process, a reactor product is withdrawn, preferably continuously, from reactor 103 via line 110.

Reactor product in line 110 may be condensed and fed to a separator 111, which, in turn, provides a vapor stream 112 and a liquid stream 113. In some embodiments, separator 111 may comprise a flasher or a knockout pot. The separator 111 may operate at a temperature from 20° C. to 250° C., e.g., from 30° C. to 225° C. or from 60° C. to 200° C. The pressure of separator 111 may be from 50 kPa to 2000 kPa, e.g., from 75 kPa to 1500 kPa or from 100 kPa to 1000 kPa.

Optionally, separator 111 may also include one or more membranes. The reactor product in line 110 may, without condensing, pass through one or more membranes to separate hydrogen and/or other non-condensable gases from the reactor product. Membranes may allow vapor separation of the reactor product. Polymer-based membranes that operate at a maximum temperature of 100° C. and at a pressure of greater than 500 kPa, e.g., greater than 700 kPa, may be used. The membranes may be palladium-based membranes that have high selectivity for hydrogen, such as palladium-based alloy with copper, yttrium, ruthenium, indium, lead, and/or rare earth metals. Suitable palladium-based membranes are described in Burkhanov, et al., “Palladium-Based Alloy Membranes for Separation of High Purity Hydrogen from Hydrogen-Containing Gas Mixtures,” Platinum Metals Rev., 2011, 55, (1), 3-12, the entirety of which is incorporated by reference. Efficient hydrogen separation palladium-based membranes generally have high hydrogen permeability, low expansion when saturated with hydrogen, good corrosion resistance and high plasticity and strength during operation at temperatures of 300° C. to 700° C. Because the reactor product may contain unreacted acid, membrane 106 should tolerate acidic conditions, e.g., a pH of less than 5, or a pH of less than 4.

Vapor stream 112 exiting separator 111 may comprise hydrogen and hydrocarbons, which may be purged and/or returned to reaction zone 101. The returned portion of vapor stream 111 may pass through compressor and may be combined with hydrogen feed line 104 and co-fed to vaporizer 107.

### Water Separation

Liquid stream 113 from separator 111 may be fed to a water separation zone 115 prior to removing any organic impurities from the reactor product. Water separation zone 115 may comprise one or more adsorption units, membranes, molecular sieves, or a combination thereof As shown in FIG. 1, water separation zone 115 may comprise one or more membranes 116 and 117. Water may be removed from a liquid reactor product using pervaporation membranes or a vapor reactor product through vapor permeation. The zeolite membranes are hydrophilic and are suitable for separating water from organic mixtures due to high selectivities for water. Commercially available zeolite membranes include NaA type zeolite membrane developed by Mitsui Engineering & Shipbuilding Co. Those membranes may also tolerate acidity up to a pH of about 3 to 4. Other suitable polymer membranes which are more acid tolerant include perfluoro membranes developed by Membrane Technologies and Research.

In FIG. 1, both membranes 116 and 117 are acid resistant and have high selectivities for water. Liquid stream 113 may be heated and passed through a compressor (not shown), which supplies a driving force, and passed through first membrane 116. An intermediate water stream 118 permeates across first membrane 116 and is fed to a second membrane 117. A first retentate stream 120 comprising ethanol may also be removed from first membrane 116. Similarly, intermediate water stream 118 passes through a compressor (not shown), which supplies a driving force, and over second membrane 117 to form a second permeate stream as shown by water stream 119 and a second retentate stream 121. In this manner, first membrane 116 removes a portion of the water and second membrane 117 recovers ethanol and other organics that may have undesirably permeated through first membrane 116. Water stream 119 may be purged from the system as necessary or recycled to another step in the process if desired. Additional membranes, in series and/or in parallel, may be added to the array as necessary to further enhance water removal. Retentate streams 120 and 121 from membranes 116 and 117, respectively, may be combined to form a dried product 122. Dried product 122 preferably comprises less water than acetic acid feed line 105. In one embodiment, dried product 112 comprises less water than the total water concentration in both acetic acid feed line 105 and optional water stream 106. One or more on-line monitors may be used to monitor the water concentration in either first retentate stream 120 or second retentate stream 121. When the water concentration exceeds the acetic acid feed line 105 and/or optional water stream 106, retentate streams may be further processed through one or more membranes to remove water.

The membrane array configuration shown in water separation zone 115 of FIG. 1 is but one of many possible array configurations that may be employed in the present invention. In another array configuration, not shown, water separation zone 115 includes two (or more) membranes in series. In this aspect, liquid stream 113 may be directed to a first membrane where a first amount of water is removed in a first permeate. The resulting retentate is then sent to a second membrane where an additional amount of water is removed in a second permeate and forms a second retentate that may be further processed with additional membranes in a similar manner and/or sent to an ethanol recovery system. Additional membranes, in series and/or in parallel, may be added to the array as necessary to further enhance water removal. Optionally, depending on the composition of the permeate or retentate streams, a portion of these streams may be recycled back, directly or indirectly, to reaction zone 101. For example if the permeate stream comprises a high concentration of acetic acid, it may be beneficial to return the permeate stream to the reactor via vaporizer. Whichever membrane array configuration is employed, in preferred embodiments, the water separation system removes at least 90%, at least 95% or at least 97%, of the water from the original stream, e.g., liquid stream 113, that is sent to the water separation system.

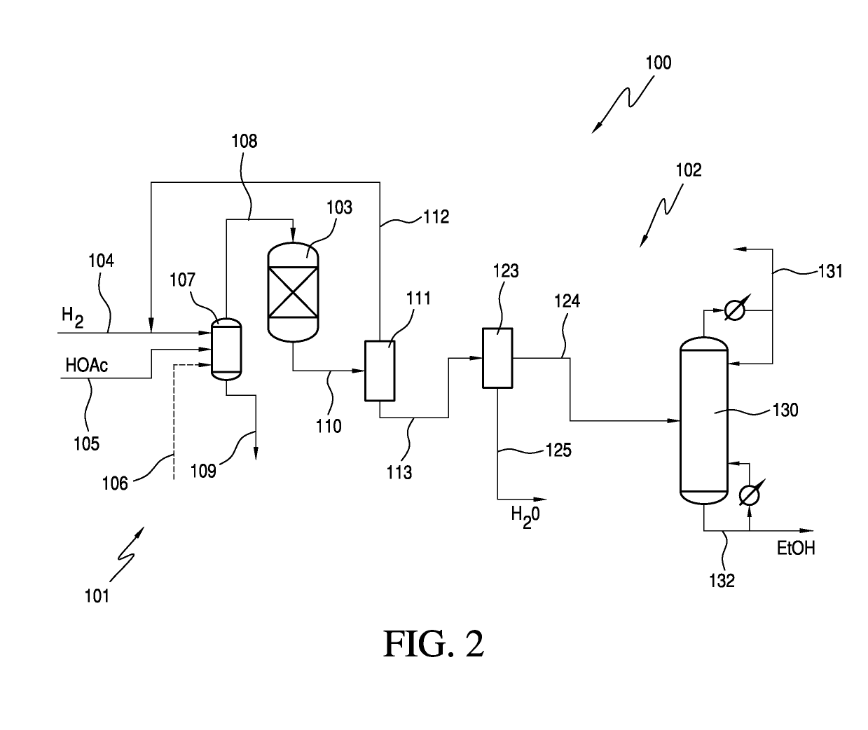

In FIG. 2, water separation zone 115 comprises a pressure swing adsorption (PSA) unit 123. PSA unit 123 is optionally operated at a temperature from 30° C. to 160° C., e.g., from 80° C. to 140° C., and a pressure from 0.01 kPa to 550 kPa, e.g., from 1 kPa to 150 kPa. PSA unit 123 may comprise two to five beds and may remove at least 95% of the water from liquid stream 113, and more preferably from 99% to 99.99% of the water from the liquid stream 113, in a water stream 125. All or a portion of water stream 125 may be purged. The remaining portion of liquid stream 113 exits PSA unit 123 as a dried product 124. Dried product 124 may have a low concentration of water of less than 10 wt. %, e.g., less than 6 wt. % or less than 2 wt. %.

Exemplary components of water stream 119 and dried product 122, 124 obtained using either the membranes or PSA are provided in Table 2 below. It should be understood that these streams may also contain additional components, not listed in Table 2.

TABLE 2
WATER SEPARATION

| | Conc. (wt. %) | Conc. (wt. %) | Conc. (wt. %) |
|---|---|---|---|
| Dried product | | | |
| Ethanol | 5 to 80 | 15 to 75 | 25 to 65 |
| Water | <5 | 0.001 to 3 | 0.01 to 1 |
| Acetic Acid | <95 | 0.001 to 75 | 0.01 to 25 |
| Ethyl Acetate | <35 | 0.001 to 25 | 0.01 to 15 |
| Acetaldehyde | <15 | 0.001 to 10 | 0.01 to 5 |
| Acetal | <10 | 0.001 to 6 | 0.01 to 4 |
| Acetone | <5 | 0.001 to 3 | 0.01 to 2 |
| Water Stream | | | |
| Acetic Acid | <10 | 0.001 to 5 | 0.01 to 2 |
| Water | 70 to 100 | 80 to 99.5 | 85 to 99 |
| Ethanol | <10 | 0.001 to 5 | 0.01 to 2 |

The amounts indicated as less than (<) in the tables throughout the present application are preferably not present and if present may be present in trace amounts or in amounts greater than 0.0001 wt. %.

### Ethanol Recovery

As shown in FIGS. 1 and 2, dried product 122, 124 is fed to a first column 130 for separating one or more organic impurities and ethanol. In one embodiment, the conversion of acetic acid is greater than 90%, e.g. greater than 95% or greater than 99%, and there are low concentrations of acetic acid. Thus, it is not necessary to separate acetic acid and the low concentrations of acetic acid would be applicable for industrial grade and/or fuel grade ethanol applications.

In first column 130, ethyl acetate and acetaldehyde are removed as a first distillate in line 131 and ethanol is removed as the first residue in line 132. First distillate in line 131 preferably is refluxed as shown, for example, at a reflux ratio from 1:30 to 30:1, e.g., from 1:10 to 10:1 or from 1:3 to 3:1. First distillate in line 131 may be recycled to reaction zone 101 or directly to reactor 103. Ethanol from dried product 122, 124 remains in the liquid phase when being recovered from first column 130. Preferably dried product 122, 124 is a liquid fed to first column 130 and ethanol is a liquid product withdrawn from first residue 132. It is noted that some of the ethanol may be volatized in first column 130, but ethanol remains in the liquid phase if it is withdrawn from first column 130 as a liquid without any condensing.

First column 130 may be a tray column or packed column. In one embodiment, first column 130 is a tray column having from 5 to 70 trays, e.g., from 15 to 50 trays or from 20 to 45 trays. First column 130 operates at a pressure ranging from 0.1 kPa to 510 kPa, e.g., from 10 kPa to 450 kPa or from 50 kPa to 350 kPa. Although the temperature of first column 130 may vary, when at about 20 kPa to 70 kPa, the temperature of the first residue exiting in line 132 preferably is from 30° C. to 75° C., e.g., from 35° C. to 70° C. or from 40° C. to 65° C. The temperature of the first distillate exiting in line 131 preferably is from 20° C. to 55° C., e.g., from 25° C. to 50° C. or from 30° C. to 45° C.

Exemplary components for the first distillate and first residue compositions for first column 130 are provided in Table 3, below. It should be understood that the distillate and residue may also contain other components, not listed in Table 3.

TABLE 3
FIRST COLUMN 130

| | Conc. (wt. %) | Conc. (wt. %) | Conc. (wt. %) |
|---|---|---|---|
| First Distillate | | | |
| Ethyl Acetate | 5 to 90 | 10 to 80 | 15 to 75 |
| Acetaldehyde | <60 | 1 to 40 | 1 to 35 |
| Ethanol | <45 | 0.001 to 40 | 0.01 to 35 |
| Water | <20 | 0.01 to 10 | 0.1 to 5 |
| First Residue | | | |
| Ethanol | 90 to 99.5 | 92 to 99.5 | 96 to 99.5 |
| Water | <10 | 0.001 to 8 | 0.01 to 4 |
| Ethyl Acetate | <1 | 0.001 to 0.05 | 0.001 to 0.01 |
| Acetic Acid | <0.5 | <0.01 | 0.001 to 0.01 |
| Acetaldehyde | <0.01 | <0.001 | Not detectable |

First residue in line 132 may contain some amounts of residual water. Depending on the desired ethanol product, it may be desired to further dry the first residue in line 132. Residual water removal may be accomplished, for example, using one or more adsorption units, membranes, molecular sieves, extractive distillation, or a combination thereof. Suitable adsorption units include pressure swing adsorption units and thermal swing adsorption units. In one embodiment, the further drying may be accomplished using a different type of water separation than used in water separation unit 115.

### Optional Reactive Distillation Column

In one optional embodiment, first column 130 may be a reactive distillation column in which acetic acid may be esterified with ethanol to form ethyl acetate. The reactive distillation column produces a distillate comprising ethyl acetate and acetaldehyde, and an ethanol residue. In one embodiment, first column 130 is a tray column having from 5 to 70 trays, e.g., from 15 to 50 trays or from 20 to 45 trays.

Optional reactive distillation column may comprise a reaction zone that contains a catalyst, such as an acidic catalyst. Suitable catalysts include, without limitation, alkyl sulfonic acids and aromatic sulfonic acids, e.g., methane sulfonic acid, benzene sulfonic acid and p-toluene sulfonic acid. Alternatively, sulfuric acid or heteropoly acids can be used within the scope of the invention. A variety of homogeneous or heterogeneous acids may also be employed within the scope of this invention.

A majority of the acetic acid in the dried product may be consumed in the optional reactive distillation column, e.g., at least 50% of the acetic acid, and more preferably at least 90%. In reacting the acetic acid, a portion of the ethanol is also consumed. Preferably less than 25% of the ethanol in the dried product is consumed, and more preferably less than 5%. The ethanol residue may comprise any remaining amounts of acetic acid, but preferably is substantially free of acetic acid, e.g., containing less than 5000 wppm acetic acid, and more preferably less than 500 wppm acetic acid. Optionally, ethanol residue may be neutralized to remove residual acetic acid. The reaction may produce additional amounts of ethyl acetate. A majority of the ethyl acetate preferably is withdrawn from optional reactive distillation column in the distillate and the water, if any, is withdrawn in the residue with the ethanol.

### Acid Separation

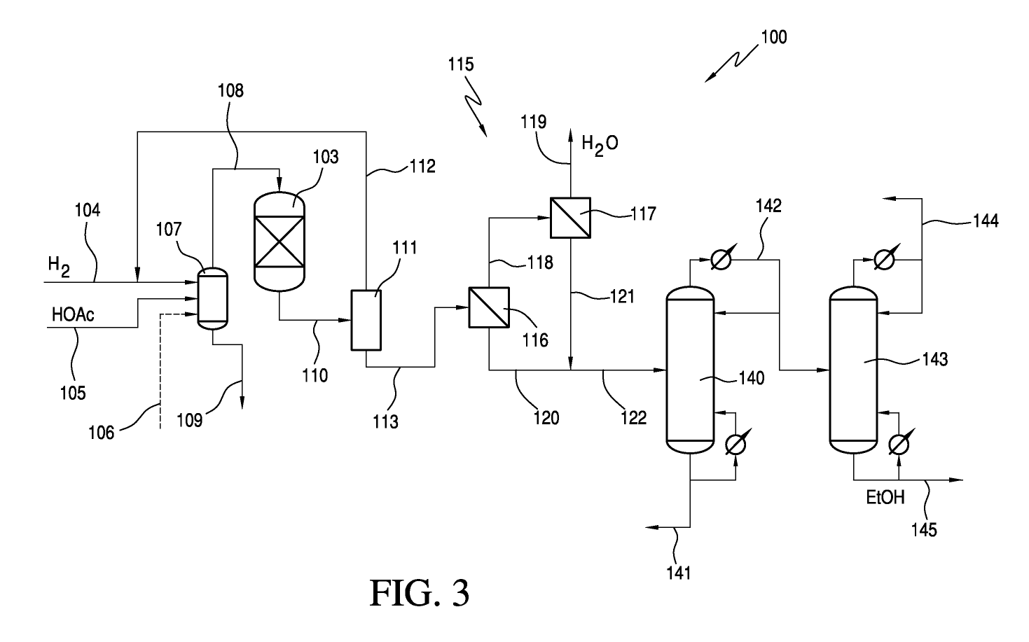

In the embodiment shown in FIG. 3, dried product 122 is introduced in a first column 140, which is also known as an “acid separation column.” The columns shown in FIG. 3 may also be used with the PSA unit 123 shown in FIG. 2. For purposes of convenience, the columns in each exemplary separation process may be referred as the first, second, third, etc., columns, but it is understood that first column 130 in FIG. 1 operates differently than the first column 140 of FIG. 3. Dried product 122 is introduced to the lower part of first column 140, e.g., lower half or lower third. In column 140, acetic acid, any remaining water, and other heavy components, if present, are removed from the dried product and are withdrawn, preferably continuously, as residue in line 141. In some embodiments, it may be preferable to withdraw a majority of the water in the residue. Acid separation column 140 also forms a first distillate, which is withdrawn in line 142, and which may be condensed and refluxed, for example, at a ratio of from 10:1 to 1:10, e.g., from 3:1 to 1:3 or from 1:2 to 2:1.

When column 140 is operated under 170 kPa pressure, the temperature of the residue exiting in line 141 preferably is from 120° C. to 150° C., e.g., from 128° C. to 142° C. or from 136° C. to 143° C. The temperature of the distillate exiting in line 142 preferably is from 85° C. to 95° C., e.g., from 85° C. to 91° C. or from 87° C. to 95° C. In some embodiments, the pressure of acid separation column 140 may range from 0.1 kPa to 510 kPa, e.g., from 1 kPa to 475 kPa or from 1 kPa to 375 kPa. Exemplary components for the distillate and residue compositions for the column 140 are provided in Table 4, below. It should be understood that the distillate and residue may also contain other components, not listed in Table 4.

TABLE 4
ACID SEPARATION COLUMN 140

| | Conc. (wt. %) | Conc. (wt. %) | Conc. (wt. %) |
|---|---|---|---|
| First Distillate | | | |
| Ethanol | 30 to 95 | 50 to 92 | 60 to 90 |
| Water | <25 | <20 | <10 |
| Acetic Acid | <1 | 0.001 to 1 | 0.01 to 0.5 |
| Ethyl Acetate | <60 | 5 to 40 | 8 to 45 |
| Acetaldehyde | <10 | 0.001 to 5 | 0.01 to 4 |
| Acetal | <4.0 | <3.0 | <2.0 |
| Acetone | <0.05 | 0.001 to 0.03 | 0.01 to 0.025 |
| First Residue | | | |
| Acetic Acid | 80 to 99.9 | 85 to 99 | 90 to 95 |
| Water | <20 | 0.001 to 15 | 0.01 to 10 |
| Ethanol | <3 | <1 | <0.05 |

Some species, such as acetals, may decompose in column 140 such that very low amounts, or even no detectable amounts, of acetals remain in the distillate or residue. In addition, an equilibrium reaction between acetic acid and ethanol or between ethyl acetate and water may occur in the reactor product after it exits reactor 103. Depending on the concentration of acetic acid in the reactor product, this equilibrium may be driven toward formation of ethyl acetate. This equilibrium may be regulated using the residence time and/or temperature of reactor product.

Depending on the amount of water and acetic acid contained in the residue of column 140, line 141 may be treated in one or more of the following processes. The following are exemplary processes for further treating first residue and it should be understood that any of the following may be used regardless of acetic acid concentration. When the residue comprises a majority of acetic acid, e.g., greater than 70 wt. %, the residue may be recycled to the reactor without any separation of the water. In one embodiment, the residue may be separated into an acetic acid stream and a water stream when the residue comprises a majority of acetic acid, e.g., greater than 50 wt. %. Acetic acid may also be recovered in some embodiments from the first residue having a lower acetic acid concentration. The residue may be separated into the acetic acid and water streams by a distillation column or one or more membranes. If a membrane or an array of membranes is employed to separate the acetic acid from the water, the membrane or array of membranes may be selected from any suitable acid resistant membrane that is capable of removing a permeate water stream. The resulting acetic acid stream optionally is returned to reactor 103. The resulting water stream may be used as an extractive agent or to hydrolyze an ester-containing stream in a hydrolysis unit.

In other embodiments, for example where residue in line 141 comprises less than 50 wt. % acetic acid, possible options include one or more of: (i) returning a portion of the residue to reactor 103, (ii) neutralizing the acetic acid, (iii) reacting the acetic acid with an alcohol, or (iv) disposing of the residue in a waste water treatment facility.

Optionally, liquid stream 113 or dried product 122 may be further fed to an esterification reactor, hydrolysis reactor, hydrogenolysis reactor, or combination thereof. An esterification reactor may be used to consume acetic acid present in the reactor product to further reduce the amount of acetic acid that would otherwise need to be removed. Hydrolysis may be used to convert ethyl acetate into acetic acid (which may be recycled to reaction zone 101) and ethanol, while hydrogenolysis may be used to convert ethyl acetate in the reactor product to ethanol.

The distillate in line 142 preferably comprises ethanol and optionally ethyl acetate, acetaldehyde, and water. The final ethanol product may be derived from the distillate in line 142. In one embodiment, the weight ratio of water in the residue to the water in the distillate is greater than 1:1, e.g., greater than 2:1 or greater than 4:1. In addition, the weight ratio of acetic acid in the residue to acetic acid in the distillate is optionally greater than 10:1, e.g., greater than 15:1 or greater than 20:1. Preferably, the distillate in line 142 is substantially free of acetic acid and may contain, if any, only trace amounts of acetic acid.

As shown in FIG. 3, there is also provided a second column 143, also referred to as a “light ends column,” that removes ethyl acetate and acetaldehyde from the first distillate in line 142. In this embodiment, column 143 produces a second distillate in line 144 comprising ethyl acetate and acetaldehyde, and a second residue in line 145 comprising ethanol.

First distillate in line 142 is introduced to the second column 143 preferably in the top part of column, e.g., top half or top third. Column 143 may be a tray column or packed column. In one embodiment, column 143 is a tray column having from 5 to 70 trays, e.g., from 15 to 50 trays or from 20 to 45 trays. As one example, when a 30 tray column is utilized in a column without water extraction, line 142 is introduced preferably at tray 2.

Optionally, light ends column 143 may be an extractive distillation column. Suitable extractive agents may include, for example, dimethylsulfoxide, glycerine, diethylene glycol, 1-naphthol, hydroquinone, N,N′-dimethylformamide, 1,4-butanediol; ethylene glycol-1,5-pentanediol; propylene glycol-tetraethylene glycol-polyethylene glycol; glycerine-propylene glycol-tetraethylene glycol-1,4-butanediol, ethyl ether, methyl formate, cyclohexane, N,N′-dimethyl-1,3-propanediamine, N,N′-dimethylethylenediamine, diethylene triamine, hexamethylene diamine and 1,3-diaminopentane, an alkylated thiopene, dodecane, tridecane, tetradecane, chlorinated paraffins, or a combination thereof. In another aspect, the extractive agent may be an aqueous stream comprising water. If the extraction agent comprises water, the water may be obtained from an external source or from an internal return/recycle line from one or more of the other columns, such as from a portion of the water stream 119. Generally, the extractive agent is fed above the entry point of distillate in line 142. When extractive agents are used, a suitable recovery system, such as a further distillation column, may be used to remove the extractive agent and recycle the extractive agent.

Although the temperature and pressure of second column 143 may vary, when at about 20 kPa to 70 kPa, the temperature of the second residue exiting in line 145 preferably is from 30° C. to 75° C., e.g., from 35° C. to 70° C. or from 40° C. to 65° C. The temperature of the second distillate exiting in line 144 preferably is from 20° C. to 55° C., e.g., from 25° C. to 50° C. or from 30° C. to 45° C. Second column 143 may operate at a reduced pressure, near or at vacuum conditions, to further favor separation of ethyl acetate and ethanol. In other embodiments, the pressure of column 143 may range from 0.1 kPa to 510 kPa, e.g., from 1 kPa to 475 kPa or from 1 kPa to 375 kPa. Exemplary components for the second distillate and second residue compositions for second column 143 are provided in Table 5, below. It should be understood that the second distillate and second residue may also contain other components, not listed in Table 5.

TABLE 5
LIGHT ENDS COLUMN 143

| | Conc. (wt. %) | Conc. (wt. %) | Conc. (wt. %) |
|---|---|---|---|
| Second Distillate | | | |
| Ethyl Acetate | 5 to 90 | 10 to 80 | 15 to 75 |
| Acetaldehyde | <60 | 1 to 40 | 1 to 35 |
| Ethanol | <45 | 0.001 to 40 | 0.01 to 35 |
| Water | <20 | 0.01 to 10 | 0.1 to 5 |
| Acetal | <5 | 0.001 to 2 | 0.01 to 1 |
| Second Residue | | | |
| Ethanol | 80 to 99.5 | 85 to 99.5 | 90 to 99.5 |
| Water | <20 | 0.001 to 15 | 0.01 to 10 |
| Ethyl Acetate | <1 | 0.001 to 2 | 0.001 to 0.5 |
| Acetic Acid | <0.5 | <0.01 | 0.001 to 0.01 |

The weight ratio of ethanol in the second residue to ethanol in the second distillate preferably is at least 2:1, e.g., at least 5:1, at least 8:1, at least 10:1 or at least 15:1. The weight ratio of ethyl acetate in the second residue to ethyl acetate in the second distillate preferably is less than 0.4:1, e.g., less than 0.2:1 or less than 0.1:1. It should be understood that when an extractive agent is used, the composition of the residue would also include the extractive agent.

Depending on the intended ethanol application, it may be desirable to remove water from the second residue in line 145. In some embodiments, removing substantially all of the water produces an anhydrous ethanol product suitable for fuel applications. Water may be removed from the second residue in line 145 using any of several different separation techniques. Particularly preferred techniques include the use of a distillation column, one or more membranes, one or more adsorption units or a combination thereof.

The second distillate in line 144, which comprises ethyl acetate and/or acetaldehyde, preferably is refluxed as shown in FIG. 3, for example, at a reflux ratio of from 1:30 to 30:1, e.g., from 1:5 to 5:1 or from 1:3 to 3:1. In one aspect, not shown, the distillate or a portion thereof may be returned to reactor 103. In some embodiments, it may be advantageous to return a portion of distillate to reactor 103. The ethyl acetate and/or acetaldehyde in the distillate may be further reacted in reactor 103 or in an additional separate reactor to produce additional ethanol before returning to reactor 103.

Any of columns described with embodiments of the present invention may comprise any distillation column capable of separation and/or purification. Each column preferably comprises a tray column having from 1 to 150 trays, e.g., from 10 to 100, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column or they may be arranged in two or more columns such that the vapor from the first section enters the second section while the liquid from the second section enters the first section, etc.

The temperatures and pressures employed in the columns may vary. As a practical matter, pressures from 10 kPa to 3000 kPa will generally be employed in these zones although in some embodiments, subatmospheric pressures or superatmospheric pressures may be employed. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column. In addition, feed rates may vary depending on the size of the production process and, if described, may be generically referred to in terms of feed weight ratios.

The associated condensers and liquid separation vessels that may be employed with each of the distillation columns may be of any conventional design and are simplified in the figures. Heat may be supplied to the base of each column or to a circulating bottom stream through a heat exchanger or reboiler. Other types of reboilers, such as internal reboilers, may also be used. The heat that is provided to the reboilers may be derived from any heat generated during the process that is integrated with the reboilers or from an external source such as another heat generating chemical process or a boiler. Although one reactor and flasher are shown, additional reactors, flashers, condensers, heating elements, and other components may be used in embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

The finished ethanol composition obtained by the processes of the present invention preferably comprises from 75 to 100 wt. % ethanol, e.g., from 80 to 99.5 wt. % or from 85 to 96 wt. % ethanol, based on the total weight of the finished ethanol composition. Exemplary finished ethanol compositional ranges are provided below in Table 6.

TABLE 6
FINISHED ETHANOL COMPOSITIONS

| Component | Conc. (wt. %) | Conc. (wt. %) | Conc. (wt. %) |
|---|---|---|---|
| Ethanol | 75 to 99.5 | 80 to 99 | 85 to 96 |
| Water | <12 | 0.01 to 9 | 0.5 to 8 |
| Acetic Acid | <1 | <0.1 | <0.01 |
| Ethyl Acetate | <2 | <0.5 | <0.05 |
| Acetal | <0.05 | <0.01 | <0.005 |
| Acetone | <0.05 | <0.01 | <0.005 |
| Isopropanol | <0.5 | <0.1 | <0.05 |
| n-propanol | <0.5 | <0.1 | <0.05 |

The finished ethanol composition of the present invention preferably contains very low amounts, e.g., less than 0.5 wt. %, of other alcohols, such as methanol, butanol, isobutanol, isoamyl alcohol and other C₄-C₂₀ alcohols. In one embodiment, the amount of isopropanol in the finished ethanol composition is from 80 to 1,000 wppm, e.g., from 95 to 1,000 wppm, from 100 to 700 wppm, or from 150 to 500 wppm. In one embodiment, the finished ethanol composition is substantially free of acetaldehyde, optionally comprising less than 8 wppm acetaldehyde, e.g., less than 5 wppm or less than 1 wppm.

In some embodiments, when further water separation is used, the ethanol product may be withdrawn as a stream from the water separation unit as discussed above. In such embodiments, the ethanol concentration of the ethanol product may be higher than indicated in Table 5, and preferably is greater than 97 wt. % ethanol, e.g., greater than 98 wt. % or greater than 99.5 wt. %. The ethanol product in this aspect preferably comprises less than 3 wt. % water, e.g., less than 2 wt. % or less than 0.5 wt. %.

The finished ethanol composition produced by the embodiments of the present invention may be used in a variety of applications including applications as fuels, solvents, chemical feedstocks, pharmaceutical products, cleansers, sanitizers, hydrogen transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, aldehydes, and higher alcohols, especially butanol. In the production of ethyl acetate, the finished ethanol composition may be esterified with acetic acid. In another application, the finished ethanol composition may be dehydrated to produce ethylene. Any known dehydration catalyst can be employed to dehydrate ethanol, such as those described in copending U.S. Pub. No. 2010/0030002 and 2010/0030001, the entire contents and disclosures of which are hereby incorporated by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Y in U.S. Pat. No. 3,130,007, the entireties of which are hereby incorporated by reference.

In order that the invention disclosed herein may be more efficiently understood, examples are provided below. It should be understood that these examples are for illustrative purposes only and are not to be construed as limiting the invention in any manner.

### EXAMPLE 1

The following examples were prepared with ASPEN Plus 7.1 simulation software to test various feed composition and separation systems.

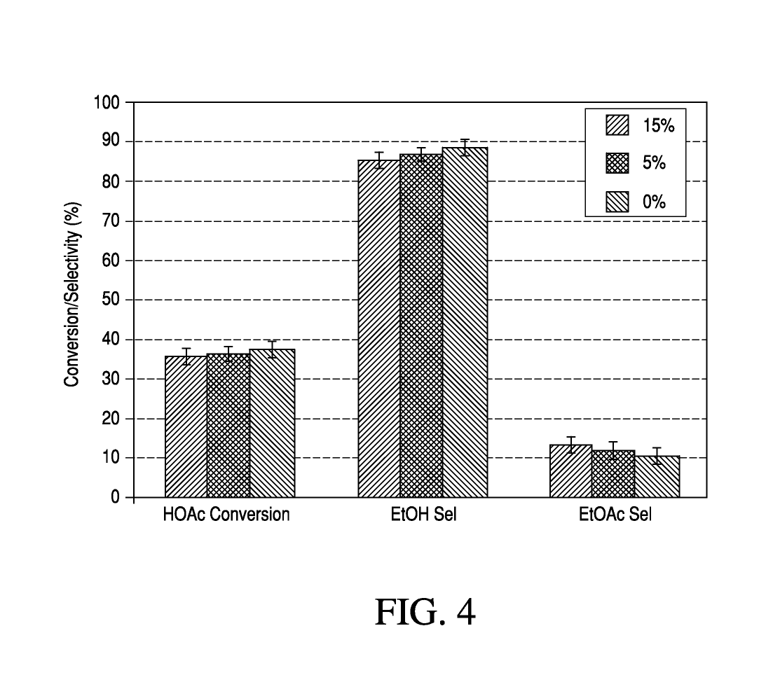

Three acetic acid feed streams were prepared comprising 0 wt. % water, 5 wt. % water and 15 wt. % water. Each feed stream was vaporized along with hydrogen and fed to a reactor. The reactor was maintained at a temperature of 250° C. and a constant pressure of about 1,820 kPa. The catalyst comprised 1.6 wt. % platinum and 1 wt. % tin supported on ⅛ inch calcium silicate modified silica extrudates. The conversion and selectivity to ethanol and ethyl acetate is shown in FIG. 4. The slight changes in conversion and selectivity shown in FIG. 4 are too small to be statistically significant and thus the presence of water does not affect conversion and selectivity in the reactor. As shown in FIG. 4, the error bar represents one standard deviation.

### EXAMPLE 2

Acetic acid having about 5 wt. % water was hydrogenated in the presence of a catalyst with a conversion rate of 90.0%. Crude product having 54.5 wt. % ethanol, 25.2 wt. % water, 10.0 wt. % acetic acid, 9.0 wt. % of ethyl acetate and 0.6 wt. % acetaldehyde was fed through an array of membranes with a selectivity for water. The permeate stream contained 100 wt. % water and the retentate stream, e.g., dried product, contained 72.9 wt. % ethanol, 13.4 wt. % acetic acid, 12.0 wt. % ethyl acetate, 0.8 wt. % acetaldehyde, 0.8 wt. % DEA, and less than 0.01 wt. % water. Thus, the dried product contained less water than the feed streams that comprise acetic acid and/or ethyl acetate.

The dried product was introduced to an acid separation column to separate into a distillate stream and a residue stream. The distillate stream comprised 84.1 wt. % ethanol, 13.9 wt. % ethyl acetate, 1.0 wt. % acetaldehyde, 0.9 wt. % DEA, and 0.1 wt. % water. The residue stream comprised 99.1 wt. % acetic acid, 0.5 wt. % ethanol, and 0.4 wt. % DEA.

While the invention has been described in detail, modifications within the spirit and scope of the invention will be readily apparent to those of skill in the art. In addition, it should be understood that aspects of the invention and portions of various embodiments and various features recited herein and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing ethanol, comprising the steps of:
providing at least one feed stream comprising water and a carbonylation stock selected from the group consisting of acetic acid, and acetic acid and an ester thereof;
hydrogenating the acetic acid and/or an ester thereof from the feed stream in a reactor to form a reactor product comprising ethanol, water, and one or more organic impurities;
separating at least a portion of the reactor product to yield a water stream and a dried product, wherein the dried product comprises less water based on total weight than the feed stream; and
separating at least a portion of the dried product to yield one or more streams comprising the one or more organic impurities and an ethanol stream comprising less than 0.01 wt. % of the one or more organic impurities.

2. The process of claim 1, wherein the feed stream is water and acetic acid.

3. The process of claim 1, wherein the one or more organic impurities are selected from the group consisting of ethyl acetate, acetic acid, acetone, diethyl acetal, diethyl ether, and acetaldehyde.

4. The process of claim 1, wherein the water stream comprises at least 90% of the water from the reactor product.

5. The process of claim 1, wherein the water stream comprises less than 5000 wppm ethanol.

6. The process of claim 1, wherein the water is removed using an adsorption unit selected from the group of pressure swing adsorption units and thermal swing adsorption units.

7. The process of claim 1, wherein the water is removed using one or more membranes.

8. The process of claim 7, wherein the one or more membranes are acid resistant and have high selectivities for permeating water.

9. The process of claim 1, wherein water is removed from the reactor product before a majority of the one or more organic impurities are removed from the reactor product.

10. The process of claim 1, wherein dried product comprises from 0.01 to 4 wt. % water.

11. The process of claim 1, further comprising
feeding the at least a portion of the dried product to a reactive distillation column; and
withdrawing a distillate comprising ethyl acetate and acetaldehyde, and a residue comprising ethanol.

12. The process of claim 11, wherein the residue comprises less than 5000 wppm acetic acid.

13. The process of claim 1, further comprising
separating at least a portion of the dried product in a first column to yield a first residue comprising acetic acid and a first distillate comprising ethanol and ethyl acetate; and
separating at least a portion of the first distillate in a second column to yield a second distillate comprising ethyl acetate and a second residue comprising ethanol.

14. The process of claim 1, wherein the acetic acid is formed from methanol and carbon monoxide, wherein each of the methanol, the carbon monoxide, and hydrogen for the hydrogenating step is derived from syngas, and wherein the syngas is derived from a carbon source selected from the group consisting of natural gas, oil, petroleum, coal, biomass, and combinations thereof.

15. A process for producing ethanol, comprising the steps of:
providing at least one feed stream comprising water and a carbonylation stock selected from the group consisting of acetic acid, and acetic acid and an ester thereof; hydrogenating the acetic acid and/or an ester thereof from the feed stream in a reactor to form a reactor product comprising ethanol, water, and one or more organic impurities;
separating at least a portion of the reactor product to yield a water stream and a dried product, wherein the dried product comprises less water based on total weight than the feed stream; and
recovering ethanol from the dried product.

16. The process of claim 15, wherein the feed stream is water and acetic acid.

17. The process of claim 15, wherein the ethanol remains in the liquid phase when recovering ethanol from the dried product.

18. The process of claim 15, wherein the one or more organic impurities are selected from the group consisting of ethyl acetate, acetic acid, acetone, diethyl acetal, diethyl ether, and acetaldehyde.

19. The process of claim 15, wherein the water stream comprises at least 90% of the water from the reactor product.

20. The process of claim 15, wherein the water stream comprises less than 5000 wppm ethanol.

21. The process of claim 15, wherein the water is removed using an adsorption unit selected from the group of pressure swing adsorption units and thermal swing adsorption units.

22. The process of claim 15, wherein the water is removed using one or more membranes.

23. The process of claim 15, further comprising separating at least a portion of the dried product in a first column to yield a first residue comprising ethanol and a first distillate comprising ethyl acetate and acetaldehyde.
